(19) United States
(12) Patent Application Publication
(10) Pub. No.: US 2025/0090808 A1
(43) Pub. Date: Mar. 20, 2025

(54) APPARATUS FOR THE INTRODUCTION AND MANIPULATION OF MULTIPLE TELESCOPING CATHETERS

(71) Applicant: EDWARDS LIFESCIENCES CORPORATION, Irvine, CA (US)

(72) Inventors: Matthew T. Winston, Aliso Viejo, CA (US); Christopher J. Olson, Laguna Hills, CA (US); Asher L. Metchik, Rolling Hills Estates, CA (US); Tri D. Tran, Fountain Valley, CA (US); Alexander J. Siegel, Irvine, CA (US)

(21) Appl. No.: 18/961,981

(22) Filed: Nov. 27, 2024

Related U.S. Application Data
(62) Division of application No. 17/992,427, filed on Nov. 22, 2022, now Pat. No. 12,178,973.
Application No. 17/992,427 is a continuation of application No. 16/870,790, filed on May 8, 2020, now Pat. No. 11,517,718.
Application No. 16/870,790 is a continuation of application No. 15/796,436, filed on Oct. 27, 2017, now Pat. No. 10,653,862.
(60) Provisional application No. 62/418,528, filed on Nov. 7, 2016.

(51) Int. Cl. A61M 25/01; A61F 2/24; A61M 25/00; A61M 25/06

(52) U.S. Cl. CPC A61M 25/0147; A61F 2/2427; A61F 2/2436; A61M 25/0662; A61F 2/2433; A61M 25/005; A61M 2025/015

(57) ABSTRACT

A steerable catheter includes a shaft and a pull wire. The shaft includes a proximal portion and a distal portion. The shaft includes an inner layer that defines an inner lumen of the shaft. The shaft comprises an outer layer that defines an outer diameter of the shaft. The shaft comprises a pull-wire lumen between the inner layer and the outer layer. The pull wire extends through the pull-wire lumen. The pull wire has a proximal end portion and a distal end portion. The distal end portion of the pull wire is fixed to the distal portion of the shaft. The proximal end portion of the pull wire extends radially outward of the outer layer. Increasing tension in the pull wire curves the distal portion of the shaft.

### CROSS REFERENCE TO RELATED APPLICATIONS

This application is a divisional of U.S. patent application Ser. No. 17/992,427, filed on Nov. 22, 2022, which is a continuation of U.S. patent application Ser. No. 16/870,790, filed on May 8, 2020, now U.S. Pat. No. 11,517,718, which is a continuation of U.S. patent application Ser. No. 15/796,436, filed Oct. 27, 2017, now U.S. Pat. No. 10,653,862, which claims the benefit of U.S. Provisional Patent Application No. 62/418,528, filed Nov. 7, 2016, which are incorporated herein by reference in their entireties for all purposes.

### FIELD

The present application pertains to embodiments of steerable endovascular delivery devices.

### BACKGROUND

Endovascular delivery devices are used in various procedures to deliver prosthetic medical devices or instruments to locations inside the body that are not readily accessible by surgery or where access without surgery is desirable. Access to a target location inside the body can be achieved by inserting and guiding the delivery device through a pathway or lumen in the body, including, but not limited to, a blood vessel, an esophagus, a trachea, any portion of the gastrointestinal tract, a lymphatic vessel, to name a few. In one specific example, a prosthetic heart valve can be mounted in a crimped state on the distal end of a delivery device and advanced through the patient's vasculature (e.g., through a femoral artery) until the prosthetic valve reaches the implantation site in the heart. The prosthetic valve is then expanded to its functional size such as by inflating a balloon on which the prosthetic valve is mounted, or by deploying the prosthetic valve from a sheath of the delivery device so that the prosthetic valve can self-expand to its functional size.

The usefulness of delivery devices is largely limited by the ability of the device to successfully navigate through small vessels and around tight bends in the vasculature, such as through the inferior vena cava or around the aortic arch. Various techniques have been employed to adjust the curvature of a section of a delivery device to help “steer” the valve through bends in the vasculature. Typically, a delivery device employs a pull wire having a distal end fixedly secured to the steerable section and a proximal end operatively connected to an adjustment knob located on a handle of the delivery device outside the body. The pull wire is typically disposed in a pull-wire lumen that extends longitudinally in or adjacent to a wall of the delivery device, for example, a sheath or catheter. Adjusting the adjustment knob, for example, rotating the knob, applies a pulling force on the pull wire, which in turn causes the steerable section to bend.

A drawback of many guide sheaths is that they are prone to undesirable deformation when deflected or flexed. For example, a guide sheath subject to significant curvature, such as when accessing the mitral valve in a transseptal approach, may kink at one or more locations along the radius of curvature, dramatically reducing the inner diameter of the guide sheath and resulting in unpredictable movement of the distal end of the guide sheath. A flexed guide sheath may also “pancake,” in which the cross-section of the catheter is ovalized due to a lack of adherence between the materials of adjacent layers of the sheath. Additionally, a flexed guide sheath may be reduced in length, or foreshortened, due to axial compression of the shaft as it is flexed. Such deformation of the guide sheath, especially at the distal end, can interfere with the precise positioning of an implant at the treatment site. Thus, a need exists for improved steerable shaft devices.

### SUMMARY

Certain embodiments of the disclosure concern delivery apparatuses with steerable shafts. In a representative embodiment, a delivery apparatus comprises a steerable shaft having a proximal portion, a distal portion, and a pull-wire conduit that extends at least partially through the proximal and distal portions of the shaft. The delivery apparatus further includes a pull wire extending through the pull-wire conduit and having a proximal end portion and a distal end portion. The distal end portion of the pull wire is fixed to the distal portion of the shaft. The delivery apparatus further comprises an adjustment mechanism operatively connected to the proximal end portion of the pull wire and configured to increase and decrease tension in the pull wire to adjust the curvature of the distal portion of the shaft. The distal portion of the shaft comprises a steerable portion having one or more layers. The steerable portion includes a compression-resistance portion incorporated into a respective layer of the steerable portion, and extending angularly along a portion of a cross-section of the layer. The layer of the steerable portion into which the compression-resistance portion is incorporated has a first hardness, and the compression-resistance portion has a second hardness that is greater than the first hardness.

In another representative embodiment, a method comprises inserting a shaft of a delivery apparatus into the body of a patient, the shaft having a proximal portion, a distal portion, and a pull-wire conduit that extends at least partially through the proximal and distal portions. A pull wire extends through the pull-wire conduit, and the distal portion of the shaft comprises a steerable portion having one or more layers. The steerable portion includes a compression-resistance portion incorporated into a respective layer of the steerable portion and extending angularly along a portion of a cross-section of the layer. The layer of the steerable portion into which the compression-resistance portion is incorporated has a first hardness, and the compression-resistance portion has a second hardness that is greater than the first hardness. The method further comprises applying tension to the pull wire to adjust the curvature of the distal portion of the shaft.

In another representative embodiment, a delivery apparatus comprises a steerable shaft having a proximal portion, a distal portion, and a pull-wire conduit that extends at least partially through the proximal and distal portions of the shaft. The delivery apparatus further includes a pull wire extending through the pull-wire conduit and having a proximal end portion and a distal end portion. The distal end portion of the pull wire is fixed to the distal portion of the shaft. The delivery apparatus further comprises an adjustment mechanism operatively connected to the proximal end portion of the pull wire and configured to increase and decrease tension in the pull wire to adjust the curvature of the distal portion of the shaft. The distal portion of the shaft comprises one or more layers and a compression-resistance portion incorporated into a respective layer of the distal portion. The compression-resistance portion extends angularly along a portion of a cross-section of the layer and has a hardness that is greater than a hardness of the layer into which the compression-resistance portion is incorporated. The compression-resistance portion is angularly offset from the pull-wire conduit along the cross-section of the layer.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a representative embodiment of a delivery apparatus.

FIG. 2 is a side elevation view of the guide sheath of the delivery apparatus of FIG. 1.

FIG. 3 is cross-sectional side elevation view of the distal portion of the guide sheath of FIG. 1 illustrating the atraumatic tip portion and the coupling portion.

FIG. 4 is a cross-sectional view of the guide sheath of FIG. 1 taken along line 4-4 of FIG. 2.

FIG. 5 is a side elevation view illustrating the pull ring and the distal portions of the pull wires coupled to the pull ring.

FIG. 6 is a plan view of the distal portion of the guide sheath of FIG. 1 illustrating the various layers of the guide sheath.

FIG. 7 is a perspective view of the distal portion of the guide sheath of FIG. 1 illustrating the compression-resistance portion of the outer layer.

FIG. 8 is a cross-sectional view of the guide sheath of FIG. 1 taken along line 8-8 of FIG. 2.

FIG. 9 is a detail view of the pull-wire conduit receiving portion of the guide sheath of FIG. 8.

FIG. 10 is a cross-sectional view of the guide sheath of FIG. 1 taken along line 10-10 of FIG. 2.

FIG. 11 is a perspective view of the guide sheath of FIG. 1 illustrating flexing of the guide sheath.

FIG. 12 is a cross-sectional view of an alternative embodiment of a guide sheath including one pull wire.

### DETAILED DESCRIPTION

In particular embodiments, a delivery apparatus that can be used to deliver a medical device, tools, agents, or other therapy to a location within the body of a subject can include one or more steerable catheters or sheaths. Examples of procedures in which steerable catheters and sheaths are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices or components thereof, including ultrasound transducers; and positioning energy sources, for example, for performing lithotripsy, RF sources, ultrasound emitters, electromagnetic sources, laser sources, thermal sources, and the like.

In some embodiments, the delivery apparatus includes a steerable shaft such as a guide sheath having one or more delivery catheters coaxially disposed within the guide sheath. In certain configurations, the delivery catheters can comprise one or more balloons at or near a distal end portion of the catheter. In some implementations, the delivery apparatus can be used to deliver a medical device through the vasculature, such as to a heart of the subject. These devices may comprise one or more eccentrically positioned pull wires configured to cause the steerable shaft to curve in a given direction, or to straighten. The steerable shaft can further comprise a steerable portion located near the distal end of the shaft including a compression-resistance portion that reduces foreshortening of the shaft and increases the degree of curvature attainable for a given pulling force applied to the shaft by the pull wires, thereby enhancing the steerability of the delivery apparatus.

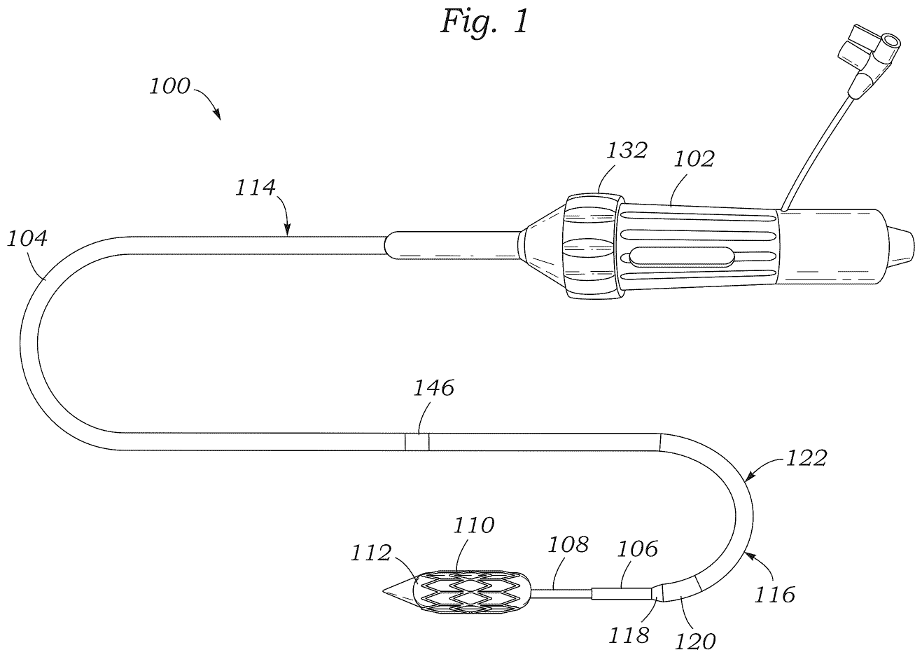

FIG. 1 illustrates a representative embodiment of a delivery apparatus 100 including a handle portion 102 and a shaft configured as a steerable guide sheath 104. The delivery apparatus 100 can be used to perform any diagnostic, therapeutic, or interventional procedure where access to a target location inside the body of a patient is desired. For example, the delivery apparatus 100 can be used to deliver and deploy a prosthetic device in the body, to deliver tools to a target location in the body, or to deliver or introduce drugs or other agents, to name a few exemplary uses.

In certain embodiments, the delivery apparatus can include one or more catheters coaxially disposed within and movable relative to the guide sheath 104. For example, in the illustrated configuration, the delivery apparatus includes an intermediate catheter configured as a steerable catheter 106 disposed within the guide sheath 104, and an inner catheter configured as a delivery or implant catheter 108 coaxially disposed within the steerable catheter 106. The implant catheter 108 can have a prosthetic device 110 mounted on a distal end of the implant catheter in a radially compressed state. In the illustrated configuration, the prosthetic device 110 is a prosthetic heart valve mounted on an inflatable balloon 112 at the distal end of the implant catheter, and the delivery apparatus can be configured to deliver the prosthetic heart valve 110 to one of the native valves of the heart (the aortic, mitral, pulmonary, or tricuspid valves).

In one specific example, the prosthetic heart valve 110 can be a plastically-expandable prosthetic heart valve, and the inflatable balloon 112 can be configured to expand and deploy the valve 110 at a treatment site. Exemplary configurations of the balloon 112 and implant catheter 108 are further disclosed in U.S. Patent Application Publication Nos. 2013/0030519, 2009/0281619, 2008/0065011, and 2007/0005131, the disclosures of which are incorporated herein by reference in their entireties. Exemplary plastically-expandable prosthetic heart valves are disclosed in U.S. Patent Application Publication Nos. 2010/0036484 and 2012/0123529, which are incorporated herein by reference.

In another example, the delivery apparatus 100 can be used to deliver and deploy a self-expandable prosthetic heart valve (e.g., a prosthetic valve having a frame formed from a shape-memory material, such as nitinol). To deliver a self-expandable prosthetic valve, the prosthetic valve can be loaded into a delivery sheath or sleeve in a radially compressed state and advanced from the distal open end of the sheath at the target location to allow the prosthetic valve to expand to its functional size. The delivery sheath can be the distal end portion of the implant catheter 108, or the distal end portion of another shaft that extends through the guide sheath 104. Further details regarding a self-expandable prosthetic valve and delivery devices for a self-expandable prosthetic valve are disclosed in U.S. Patent Application Publication Nos. 2010/0049313 and 2012/0239142, which are incorporated herein by reference. Additionally, it should be understood that the delivery apparatus 100 can be used to deliver any of various other implantable devices, such as docking devices, leaflet clips, etc.

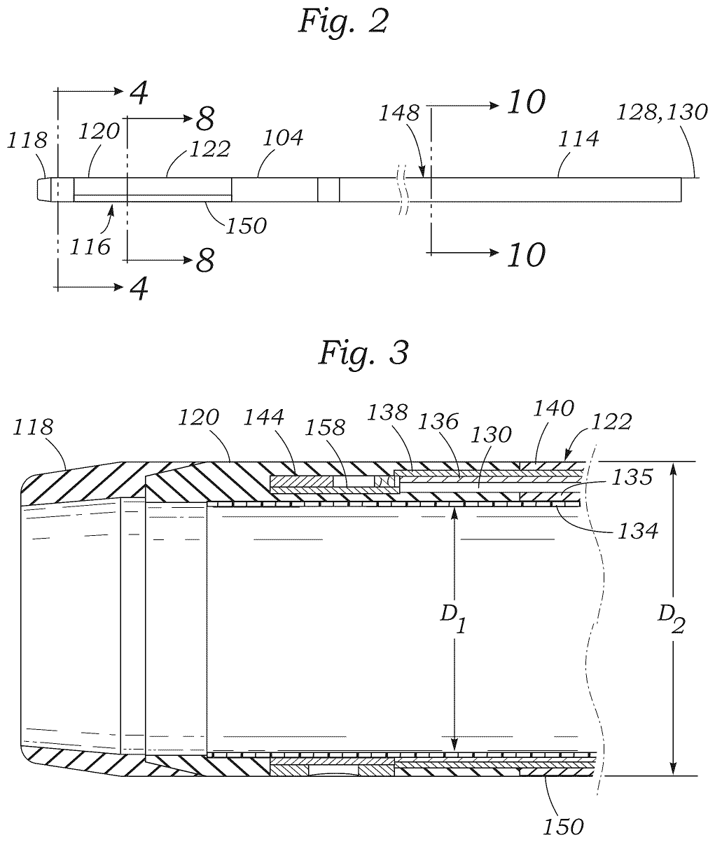

Referring to FIGS. 1 and 2, the steerable guide sheath 104 can include a proximal portion 114 coupled to the handle portion 102, and a distal portion 116. The distal portion 116 can include low durometer atraumatic tip portion 118 coupled to a coupling portion 120 positioned proximally of the atraumatic tip 118. In certain configurations, the atraumatic tip 118 can be radiopaque. The distal portion 114 of the guide sheath 104 can include a steerable portion 122 located proximally of the coupling portion 120 and configured to flex and unflex to adjust the curvature of the distal portion of the guide sheath, as described in detail below.

FIGS. 3-10 illustrate the construction of the guide sheath 104, and particularly of the distal portion 116, in greater detail. The curvature of the guide sheath 104 can be controlled by one or more eccentrically-positioned pull wires (see, e.g., FIGS. 3, 5, and 8). For example, in the illustrated configuration the guide sheath 104 includes two pull wires 124, 126 extending longitudinally through respective pull-wire lumens or conduits 128, 130. The assembled pull wires 124, 126 and conduits 128, 130 can be disposed in a pull-wire conduit portion 154 of the guide sheath. In the illustrated configuration, the pull-wire conduit portion 154 is at least partially defined by a recess 142 of an inner layer 134 of the guide sheath. In the illustrated configuration, the recess 142 can extend into an inner diameter D1 of the guide sheath 104, although other configurations are possible. In certain embodiments, the pull-wire conduits 128, 130 can be made from a lubricious material, such as polytetrafluoroethylene (PTFE) to reduce friction between the pull wires 124, 126 and the respective conduits 128, 130 as the pull wires move within the conduits.

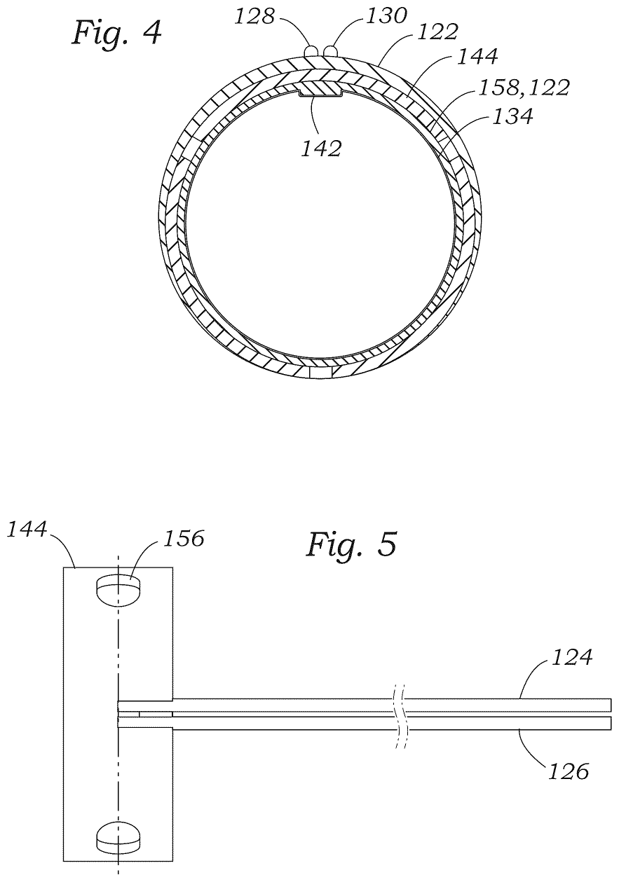

The pull wires 124, 126 can be coupled at one end to a pull ring 144 embedded in the coupling portion 120, and coupled at the opposite end to a control mechanism configured as a rotatable knob 132 of the handle 102 (see FIG. 1). Rotation of the knob 132 can increase and decrease tension in the pull wires 124, 126 which, in turn, can cause the distal portion 116, and particularly the steerable portion 122, to flex and unflex to control the curvature of the guide sheath. A cross-sectional view of the coupling portion 122 illustrating the pull ring 144 encapsulated in the coupling portion is shown in FIG. 4.

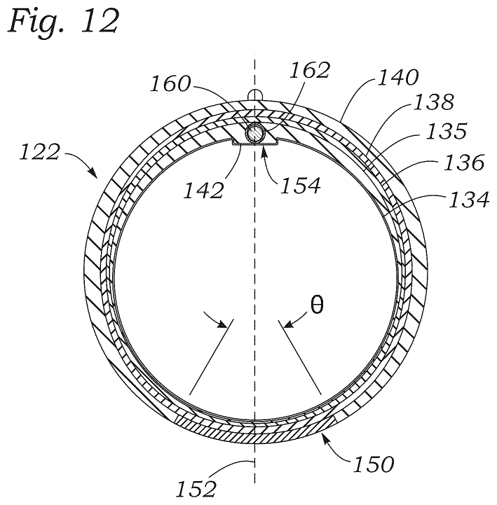

The pull ring 144 and the distal portions of the pull wires 124, 126 are shown in isolation in FIG. 5. In the illustrated configuration, the pull ring 144 can define a plurality of openings 156 about its circumference. During fabrication of the guide sheath 104, the polymeric material of the coupling portion 120 can be reflowed over the pull ring 144, and the material can flow through the openings 156 to encapsulate the pull ring in the coupling portion, as shown in FIG. 3. Additionally, although the illustrated embodiment includes two pull wires 124, 126, it should be understood that other configurations are possible. For example, the guide sheath 104 can include any suitable number of pull wires having any suitable size or layout, including a single pull wire (see FIG. 12), or more than two pull wires, depending upon the requirements of the device. The particular embodiment illustrated herein includes two pull wires because, in some configurations, two pull wires can occupy a smaller cross-sectional area than that of a single larger pull wire for transmitting a given force to the pull ring 144, particularly when relatively large forces are required (such as when flexing the guide sheath loaded with the steerable catheter 106 and the implant catheter 108).

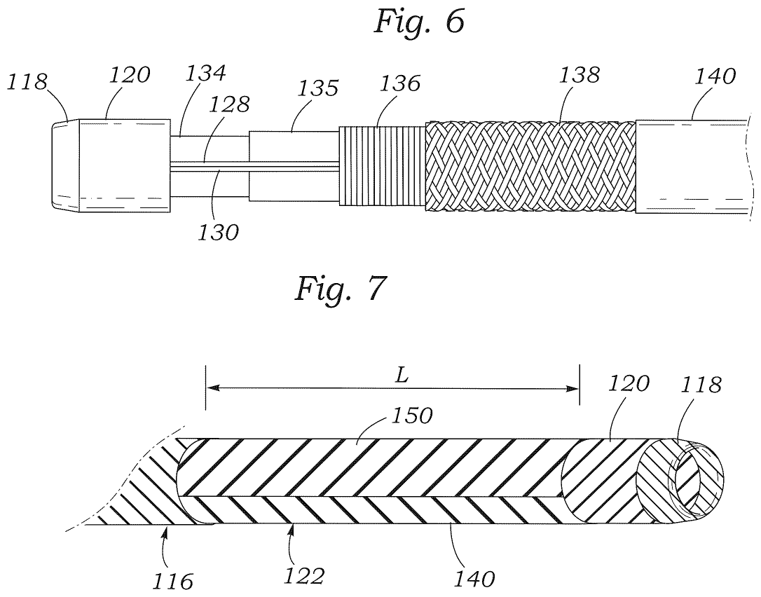

Referring to FIGS. 3-10, the guide sheath 104 can include a plurality of layers comprising a variety of different materials at different locations along the length of the guide sheath and configured to impart various properties to the guide sheath. For example, with reference to FIGS. 3 and 6, the steerable portion 122 of the guide sheath 104 comprises a first inner layer 134 defining an inner diameter D1 of the guide sheath 104, and a second pull-wire conduit encapsulating layer 135 disposed radially outward of the inner layer 134. A third helically coiled layer 136 extends over the pull-wire conduit encapsulating layer 135. A fourth braided layer 138 is disposed over the helically coiled layer 136, and a fifth outer layer 140 is disposed over the braided layer, and defines an outer diameter D2 of the guide sheath. FIG. 6 illustrates a plan view of the distal portion 116 of the guide sheath 104 with each of the outer layer 140, the braided layer 138, the helically coiled layer 136, and the pull-wire conduit encapsulating layer 135 shown partially removed to illustrate the construction of the guide sheath.

The first layer 134 extends along the full length of the guide sheath 104, and can be made from (or coated with) a lubricious material (e.g., PTFE) to allow the steerable intermediate catheter 106 to slide relative to the guide sheath 104 within the guide sheath's lumen. As stated above, the first layer 134 can also define the recess 142 of the pull-wire conduit portion 154 in which the pull wires 124, 126 and conduits 128, 130 are received.

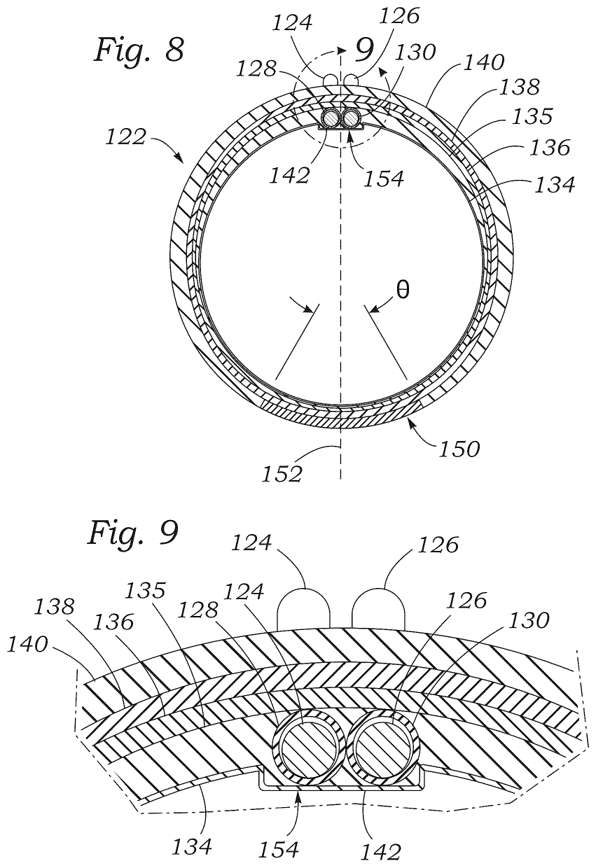

The pull-wire conduit encapsulating layer 135 can be disposed between the first inner layer 134 and the helically coiled layer 136, and can have a thickness that varies angularly around the circumference of the guide sheath. For example, with reference to FIGS. 8 and 9, the portion of the pull-wire conduit encapsulating layer 135 proximate the pull-wire conduit portion 154 can be sufficiently thick such that the layer 135 encapsulates the pull-wire conduits 128, 130 in the pull-wire conduit portion. Meanwhile, the portion of the pull-wire conduit encapsulating layer 135 opposite the pull-wire conduit portion 154 can be relatively thin. Alternatively, the pull-wire conduit encapsulating layer 135 can extend around only a portion of the cross-section of the guide sheath, such as around the portion (e.g., half) including the pull-wire conduits 128, 130. In such a configuration, the helically coiled layer 136 can directly contact the inner layer 134 along the portion of the inner layer's cross-section that is opposite the pull-wire conduits, and can transition over the pull-wire conduit encapsulating layer 135 at the location along the circumference of the inner layer 134 where the pull-wire conduit encapsulating layer originates. In some embodiments, the pull-wire conduit encapsulating layer 135 can be made from any suitable polymer, such as any of various polyether block amides (e.g., Pebax®). In certain configurations, the pull-wire conduit encapsulating layer 135 can extend from the proximal end of the coupling portion 120, through the steerable portion 122, to the pull wire exit 148 (FIG. 2).

The helically coiled layer 136 can be formed from, for example, a wire helically wrapped or wound about the pull-wire conduit encapsulating layer 135 or the first layer 134. In the illustrated embodiment, the helically coiled layer 136 can extend from adjacent the pull ring 144 proximally through the coupling portion 120 and the steerable portion 122 to a transition region 146 (FIG. 1) located between the proximal and distal portions 114, 116. In certain embodiments, the transition region 146 where the helically coiled layer 136 ends can be the location at which the outer layer 140 changes from a material having a relatively higher durometer or hardness (e.g., 63D Pebax®) to a material having a relatively lower durometer (e.g., a polyamide such as VESTAMID®). In some embodiments, gradually varying (e.g., stepwise) the hardness of the outer layer 140 or the other layers of the shaft along their length can reduce the likelihood of kinking, fracture, or warping of the shaft during flexing, or when traversing vessels of the body. Additionally, in certain examples, the helically coiled layer 136 can be made from stainless steel or titanium flat wire wound at, for example, 50 coils per inch with a pitch of 0.020 inch, and can be configured to resist kinking or crushing of the guide shaft 104, and particularly of the steerable portion 122, when it is flexed.

The braided layer 138 can extend over the helically coiled layer 136. In the illustrated configuration, the braided layer 138 can extend from the coupling portion 120 proximate the pull ring 144 proximally to, for example, the pull wire exit 148. The braided layer 138 can be, for example, metal wires braided together in a pattern to form a tubular layer over the helically coiled layer 136. For example, in the illustrated embodiment the braided layer 138 is made from stainless steel or titanium flat wires braided in an over 1 under 1 pattern, although any suitable braid pattern can be used. For example, in another representative embodiment, the wires of the braided layer 138 can be braided in a 1 over 2, under 2 pattern with a pick count of 60 picks per inch (PPI). The braided layer 138 can be configured to, for example, resist undesirable torsional deformation of the guide sheath 104 to allow the guide sheath to transmit torque, which can aid in positioning the implant at the treatment site. The braided layer 138 can also provide crush or kink-resistance properties to the guide sheath 104. In the illustrated configuration, the coupling portion 120 can also include a braided layer 158 disposed beneath the pull ring 144, as shown in FIGS. 3 and 4.

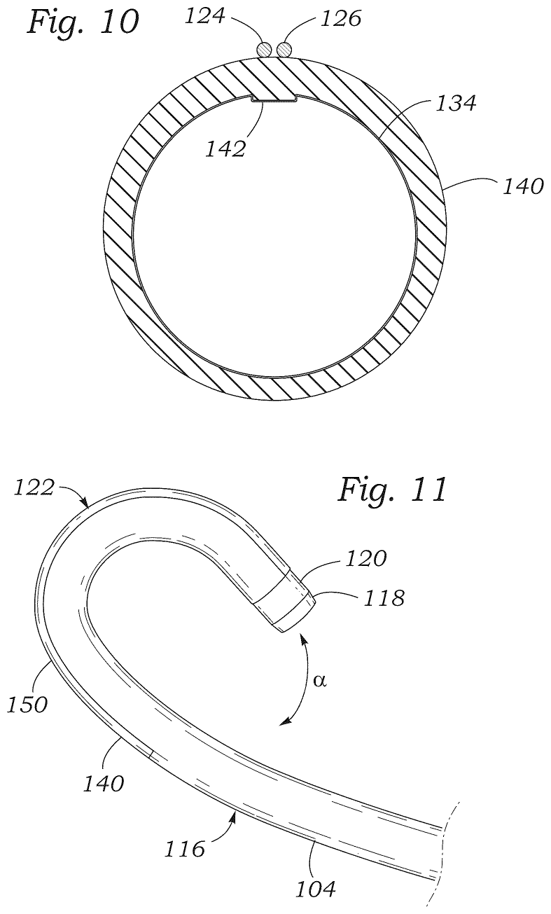

The outer layer 140 can comprise, for example, any of a variety of polymeric materials such as polyamides (e.g., VESTAMID®), polyether block amides (e.g., Pebax®), nylon, or any other suitable biocompatible polymer or combinations thereof along its length. In the illustrated configuration, the pull-wire conduit encapsulating layer 135, the helically coiled layer 136, and the braided layer 138 can terminate distally of the proximal end of the guide sheath 104. For example, in some configurations these layers can terminate at the pull wire exit 148. Proximally of the pull wire exit 148, the outer layer 140 can increase in thickness to maintain a substantially uniform outer diameter along the length of the guide sheath, as illustrated in FIG. 10.

Referring to FIGS. 7 and 8, the distal portion 116 of the guide sheath 104 can include a compression-resistance portion 150. In the illustrated configuration, the compression-resistance portion 150 is incorporated into the outer layer 140, and forms a respective part of the steerable section 122. As illustrated in FIG. 7, the compression-resistance portion 150 can extend along a length L of the steerable portion 122. The compression-resistance portion 150 can also extend circumferentially or angularly along, or occupy a respective portion of, the cross-section of the outer layer 140. For example, with reference to FIG. 8, the angular extent of the compression-resistance portion 150 along the cross-section of the outer layer 140 is denoted by the angle θ. In some embodiments, the angle θ can be from 10 degrees to 180 degrees (or half of the circumference of the cross-section). In some embodiments, the angle θ can be from 10 degrees to 90 degrees. In the embodiment of FIG. 8, the angle θ is 60 degrees.

In certain configurations, the compression-resistance portion 150 can be disposed opposite the pull wire conduits 128, 130. For example, in the illustrated configuration, the compression-resistance portion 150 is angularly offset from the pull wire conduit portion 154 by 180 degrees such that it is located diametrically opposite the pull wire conduits 128, 130. In this configuration, a plane 152 that bisects the pull wire conduit portion 154 also bisects the compression-resistance portion 150, as shown in FIG. 8. In the configuration of FIG. 8 including two pull wires and conduits, the plane 152 bisecting the pull-wire conduit portion 154 passes between the respective conduits 128, 130. However, in configurations including a single pull wire, such as the alternative configuration illustrated in FIG. 12, a single pull wire 160 and conduit 162 can be coaxially aligned with the pull-wire conduit portion 154 such that the plane 152 bisecting the pull-wire conduit portion 154 and the compression-resistance portion 150 also bisects the pull wire 160 and the conduit 162. In other configurations, the compression-resistance portion 150 can be angularly offset from the pull-wire conduit portion 154 along the cross-section of the outer layer 140 by, for example, from 90 degrees to 180 degrees, as desired.

The compression-resistance portion 150 can be made from a material having a relatively higher hardness or durometer than the remainder of the outer layer 140 in the steerable portion 122 in which the compression-resistance portion is incorporated. For example, in certain embodiments the compression-resistance portion 150 can have a durometer that is from 1.5 times to 5 times greater than a durometer of the remainder of the outer layer 140 in the steerable portion 122. In some embodiments, the durometer of the compression-resistance portion 150 can be from 2 times to 3 times greater than the durometer of the remainder of the outer layer 140 in the steerable portion 122. In an exemplary embodiment, the compression-resistance portion 150 can be made from PEBAX® having a durometer of 72D, and the remainder of the outer layer 140 of the steerable portion 122 can be made from PEBAX® having a durometer of 25D, such that a ratio of the durometer of the compression-resistance portion 150 and the durometer of the remainder of the outer layer 140 in the steerable portion 122 is 2.9:1. In some embodiments, the ratio of the durometer of the compression-resistance portion 150 to the durometer of the remainder of the outer layer 140 in the steerable portion 122 can be 3:1.

In other embodiments, the compression-resistance portion 150 can be made of any of various materials exhibiting suitable hardness properties, including metals such as stainless steel, titanium, nickel titanium alloys such as nitinol, cobalt chromium, or other polymers. In addition, in certain configurations, the compression-resistance portion need not have a thickness equal to the overall thickness of the outer layer 140. For example, the compression-resistance portion 150 can have a thickness that is less than the overall thickness of the outer layer, and may be encapsulated within the outer layer, as desired. The durometer of the compression-resistance portion 150 can also vary along its length. For example, the proximal portion of the compression-resistance portion 150 can have a relatively lower durometer than the distal portion, or vice versa.

The compression-resistance portion 150 can provide a variety of advantageous characteristics to the steerable portion 122 of the guide sheath 104. For example, the relatively higher durometer of the compression-resistance portion 150 can provide axial strength to the steerable portion 122. This can significantly reduce or prevent undesirable foreshortening of the guide sheath 104, and particularly of the steerable portion 122, when the guide catheter is flexed. More particularly, the compression-resistance portion 150 can reduce axial compression of the guide sheath and associated wrinkling of the material when the guide sheath is flexed compared to when it is in a non-deflected state. Such axial compression and wrinkling of the material can decrease the length of the guide sheath 104 as the material is deformed, and can damage the guide sheath. By reducing or eliminating foreshortening of the guide sheath 104 when it is flexed, the compression-resistance portion 150 can reduce the need for the operator to longitudinally reposition the delivery apparatus (e.g., by advancing or retracting the delivery apparatus through the patient's vasculature) in order to obtain or regain a desired position of the implant at the treatment site after flexing the guide sheath.

Additionally, the location of the compression-resistance portion 150 angularly offset from the pull wire conduits 128, 130 can aid in initiating deformation of the steerable portion 122 of the guide sheath in a specified direction. For example, when the compression-resistance portion 150 is located opposite the pull wire conduits 128, 130, the axial stiffness of the compression-resistance portion can induce deflection of the steerable portion 122 in a direction away from the compression-resistance portion when the guide sheath is flexed, as illustrated in FIG. 11. The compression-resistance portion 150 can also reduce or prevent ovalizing (also referred to as “pancaking”) of the guide sheath by reducing longitudinal movement of the different layers of the sheath relative to one another when the sheath is flexed, especially in cases in which one or more constituent layers (e.g., PTFE layers such as inner layer 134) are not strongly adhered to the surrounding layer(s).

The compression-resistance portion 150 can also increase the degree of flexion of the distal portion 116 attainable for a given force applied to the distal portion by the pull wires 124, 126, without damaging the guide sheath. The angle of flexion of the distal portion 116 is denoted a, and is illustrated in FIG. 11. For example, by reducing foreshortening of the guide sheath 104, a greater proportion of the force applied by the pull wires 124, 126 is available to flex the guide sheath instead of elastically compressing the guide sheath. Additionally, the compression-resistance portion 150 can reduce or prevent slackening of the pull wires 124, 126 attendant to foreshortening of the guide sheath 104 when it is flexed, resulting in a greater degree of curvature attainable for a given pull wire travel as compared to typical guide sheaths. As used herein, the term “pull wire travel” refers to the linear distance that a given point along the length of a pull wire moves with respect to a stationary reference (e.g., a pull wire conduit) when tension is applied to the pull wire.

Additionally, the compression-resistance portion 150, together with the helically coiled layer 136, and the braided layer 138 described above, can provide significant synergistic advantages that improve the performance of the guide sheath 104 over known steerable sheaths and catheters. For example, the distal portion of an unloaded guide sheath (e.g., a guide sheath without a delivery catheter or other shaft extending through its lumen) having an inner diameter of 22 Fr and including the compression-resistance portion, helically coiled layer, and braided layer features is capable of flexing nearly 355 degrees without kinking, and without significant foreshortening, under a force of 175 N applied by the pull wires. In this example, 50 mm of pull wire travel were required to apply a force of 175 N to the distal portion of the guide sheath. In contrast, for a typical steerable catheter device without the compression-resistance portion and without a delivery catheter or other sheath extending through its lumen, a force of 175 N produces 270 degrees of flexure and requires 60 mm of pull wire travel, and the guide sheath can be expected to foreshorten by 6 mm to 10 mm.

In another example, the distal portion of a guide sheath having an inner diameter of 22 Fr and including the above compression-resistance portion, helically coiled layer, and braided layer features, and loaded with a delivery catheter and an implant catheter extending coaxially within the lumen of the guide sheath, was capable of flexing 270 degrees without kinking, and without significant foreshortening, under a force of 250 N applied by the pull wires. In this example, 40 mm of pull wire travel were required to apply a force of 250 N to the distal portion of the guide sheath. In contrast, for a steerable catheter device without the compression-resistance portion and loaded with a delivery catheter and an implant catheter, a force of 250 N produces 180 degrees of flexure and requires 70 mm of pull wire travel, and the guide sheath can be expected to foreshorten by 6 mm to 10 mm.

In use, the delivery apparatus 100 can be introduced and advanced through the patient's vasculature using any known delivery technique. In a transfemoral procedure, the delivery apparatus can be inserted through a femoral artery and the aorta to access the heart (typically, but not exclusively used for aortic valve replacement). In a transeptal procedure (typically used for aortic or mitral valve replacement), the delivery device can be advanced to the right atrium, such as via a femoral vein, and through the septum separating the right and left ventricles. The disclosed embodiments can be particularly useful for delivering a prosthetic valve to the native mitral valve, as the torqueability of the guide sheath 104 and the relatively high degree of curvature achievable with the distal portion 116 allows for precise positioning of the prosthetic valve at the target site despite the tortuous pathway the delivery apparatus must follow to access the mitral valve in some approaches. In a transventricular procedure, the delivery apparatus can be inserted through a surgical incision made on the bare spot on the lower anterior ventricle wall (typically, but not exclusively used for aortic or mitral valve replacement). In a transatrial procedure, the delivery apparatus can be inserted through a surgical incision made in the wall of the left or right atrium. In a transaortic procedure, the delivery apparatus can be inserted through a surgical incision made in the ascending aorta and advanced toward the heart (typically, but not exclusively used for aortic valve replacement).

In certain of these procedures, the combination of the compression-resistance portion 150, the helically coiled layer 136, and the braided layer 138 can aid in precisely positioning a prosthetic device, such as the heart valve 110, at a treatment site. For example, in a transseptal procedure to access the mitral valve, after the distal end of the delivery apparatus is advanced to the treatment site, the distal portion 116 of the guide sheath 104 can be flexed to axially align the prosthetic valve 110 with the mitral valve (e.g., 180 degrees or more, in certain examples). While the distal portion 116 is in a flexed state, the guide sheath 104 can also be torqued to radially position the prosthetic valve 110 with respect to the mitral valve. The combination of the compression-resistance portion 150, the helically coiled layer 136, and the braided layer 138 can allow the guide sheath to flex without significant foreshortening or kinking, and to be torqued without undesirable torsional deformation of the shaft or associated unpredictable rotational motion of the guide sheath.

It should be understood that in alternative configurations, the components of the disclosed delivery apparatus embodiments can be rearranged without departing from the spirit of the disclosure. For example, the locations of the helically coiled layer 136 and the braided layer 138 can be reversed such that the helically coiled layer is on top of the braided layer. Alternatively, the helically coiled layer 136 and the braided layer 138 can be separated from one another by one or more intermediate layers. Additionally, the compression-resistance portion 150 need not be a respective portion of the outer layer 140, but can be incorporated into any suitable layer in the guide sheath 104. The compression-resistance portion 150 also need not extend along the entire length of the steerable portion 122, but can extend along any suitable portion of the steerable portion. The disclosed compression-resistance portion, helically coiled layer, and braided layer features described herein can also be applicable to other types of steerable catheter devices, such as delivery catheters.

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of the valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

Unless otherwise indicated, all numbers expressing quantities of components, distances, forces, ratios, angles, percentages, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited. Furthermore, not all alternatives recited herein are equivalents.

In view of the many possible embodiments to which the principles of the disclosed technology may be applied, it should be recognized that the illustrated embodiments are only preferred examples and should not be taken as limiting the scope of the disclosure. Rather, the scope of the disclosure is at least as broad as the following claims.

What is claimed is:

1. A delivery apparatus, comprising:
a steerable shaft comprising a proximal portion and a distal portion;
wherein the steerable shaft comprises an inner layer that defines an inner lumen of the shaft;
wherein the steerable shaft comprises an outer layer that defines an outer diameter of the shaft;
wherein the steerable shaft comprises a pull-wire lumen between the inner layer and the outer layer;
a pull wire extending through the pull-wire lumen and having a proximal end portion and a distal end portion,
wherein the distal end portion of the pull wire is fixed to the distal portion of the shaft;
wherein the proximal end portion of the pull wire extends radially outward of the outer layer;
an adjustment mechanism operatively connected to the proximal end portion of the pull wire; and
wherein the adjustment mechanism is configured to increase and decrease tension in the pull wire to adjust a curvature of the distal portion of the shaft.

2. The delivery apparatus of claim 1 further comprising a pull-wire lumen encapsulating layer between the inner layer and the outer layer.

3. The delivery apparatus of claim 1, wherein the inner layer defines a recess configured to receive the pull wire.

4. The delivery apparatus of claim 1 further comprising a pull-wire conduit disposed in the pull wire lumen.

5. The delivery apparatus of claim 1 further comprising a second pull wire disposed in the pull wire lumen.

6. The delivery apparatus of claim 5 wherein a proximal end portion of the second pull wire extends radially outward of the outer layer.

7. The delivery apparatus of claim 1 wherein the inner layer has a thickness that varies angularly around the steerable shaft.

8. A system, comprising:
a delivery apparatus comprising:
a steerable guide sheath comprising a proximal portion and a distal portion;
wherein the guide sheath comprises an inner layer that defines an inner lumen of the guide sheath;
wherein the steerable guide sheath comprises an outer layer that defines an outer diameter of the guide sheath;
wherein the steerable guide sheath comprises a pull-wire lumen between the inner layer and the outer layer;
a pull wire extending through the pull-wire lumen and having a proximal end portion and a distal end portion,
wherein the distal end portion of the pull wire is fixed to the distal portion of the guide sheath;
wherein the proximal end portion of the pull wire extends radially outward of the outer layer;
an adjustment mechanism operatively connected to the proximal end portion of the pull wire;
wherein the adjustment mechanism is configured to increase and decrease tension in the pull wire to adjust a curvature of the distal portion of the guide sheath;
an implant catheter coaxially disposed within the guide sheath; and
a prosthetic valve or valve repair device mounted on a distal end of the implant catheter.

9. The system of claim 8 further comprising a pull-wire lumen encapsulating layer between the inner layer and the outer layer.

10. The system of claim 8, wherein the inner layer defines a recess configured to receive the pull wire.

11. The system of claim 8 further comprising a pull-wire conduit disposed in the pull wire lumen.

12. The system of claim 8 further comprising a second pull wire disposed in the pull wire lumen.

13. The system of claim 12 wherein a proximal end portion of the second pull wire extends radially outward of the outer layer.

14. The system of claim 8 wherein the inner layer has a thickness that varies angularly around the steerable guide sheath.

15. A steerable catheter, comprising:
a shaft comprising a proximal portion and a distal portion;
wherein the shaft comprises an inner layer that defines an inner lumen of the shaft;
wherein the shaft comprises an outer layer that defines an outer diameter of the shaft;
wherein the shaft comprises a pull-wire lumen between the inner layer and the outer layer;
a pull wire extending through the pull-wire lumen and having a proximal end portion and a distal end portion,
wherein the distal end portion of the pull wire is fixed to the distal portion of the shaft;
wherein the proximal end portion of the pull wire extends radially outward of the outer layer; and
wherein increasing tension in the pull wire curves the distal portion of the shaft.

16. The steerable catheter of claim 15 further comprising a pull-wire lumen encapsulating layer between the inner layer and the outer layer.

17. The steerable catheter of claim 15, wherein the inner layer defines a recess configured to receive the pull wire.

18. The steerable catheter of claim 15 further comprising a pull-wire conduit disposed in the pull wire lumen.

19. The steerable catheter of claim 15 further comprising a second pull wire disposed in the pull wire lumen.

20. The steerable catheter of claim 19 wherein a proximal end portion of the second pull wire extends radially outward of the outer layer.
